(19) United States
(12) Patent Application Publication
(10) Pub. No.: US 2007/0122486 A1
(43) Pub. Date: May 31, 2007

(54) Nanoparticulate insulin

(75) Inventors: Simon McGurk, King of Prussia, PA (US); Elaine Liversidge, West Chester, PA (US); Daniel O'Mahoney, Dublin (IE); Amy Weiderhold, North Wales, PA (US); Araz Raoof, Dublin (IE)

Correspondence Address: ELAN DRUG DELIVERY, INC.;C/O FOLEY & LARDNER LLP, 3000 K STREET, N.W., SUITE 500, WASHINGTON, DC, 20007-5109, US

(73) Assignee: Elan Pharma International Limited

(21) Appl. No.: 11/698,151

(22) Filed: Jan. 26, 2007

Related U.S. Application Data
(63) Continuation of application No. 10/246,751, filed on Sep. 19, 2002.
(60) Provisional application No. 60/323,459, filed on Sep. 19, 2001.

(51) Int. Cl. A61K 38/28; A61K 9/14

(52) U.S. Cl. 424/489; 514/3; 977/906

(57) ABSTRACT

Nanoparticulate insulin compositions having effective an average particle size of less than about 5 microns are described. The compositions exhibit retention of insulin bioactivity, increased bioavailability, and increased consistency of bioavailability as compared to prior macro-sized insulin compositions. Methods of making nanoparticulate insulin compositions, dosage forms containing them, and the use of such formulations in insulin therapy are also described.

### CROSS-REFERENCE TO RELATED APPLICATIONS

This application is a continuation of U.S. application Ser. No. 10/246,751, filed Sep. 19, 2002, which claims priority from Provisional Patent Application No. 60/323,459, filed Sep. 19, 2001, the contents of these applications are incorporated herein by reference.

### FIELD OF THE INVENTION

The present invention is directed to nanoparticulate compositions comprising insulin, having adsorbed to the surface of the insulin particles at least one surface stabilizer.

### BACKGROUND OF THE INVENTION

A. Background Regarding Nanoparticulate Compositions

Nanoparticulate compositions, first described in U.S. Pat. No. 5,145,684 (“the '684 patent”), are particles consisting of a poorly soluble active agent having adsorbed onto the surface thereof a non-crosslinked surface stabilizer. The '684 patent also describes methods of making such nanoparticulate compositions. Nanoparticulate compositions are desirable because with a decrease in particle size, and a consequent increase in surface area, a composition is rapidly dissolved and absorbed following administration. The '684 patent does not teach or suggest nanoparticulate compositions comprising peptides or insulin.

Methods of making nanoparticulate compositions are described, for example, in U.S. Pat. Nos. 5,518,187 and 5,862,999, both for “Method of Grinding Pharmaceutical Substances;” U.S. Pat. No. 5,718,388, for “Continuous Method of Grinding Pharmaceutical Substances;” and U.S. Pat. No. 5,510,118 for “Process of Preparing Therapeutic Compositions Containing Nanoparticles.”

Nanoparticulate compositions are also described, for example, in U.S. Pat. No. 5,298,262 for “Use of Ionic Cloud Point Modifiers to Prevent Particle Aggregation During Sterilization;” U.S. Pat. No. 5,302,401 for “Method to Reduce Particle Size Growth During Lyophilization;” U.S. Pat. No. 5,318,767 for “X-Ray Contrast Compositions Useful in Medical Imaging;” U.S. Pat. NO. 5,326,552 for “Novel Formulation For Nanoparticulate X-Ray Blood Pool Contrast Agents Using High Molecular Weight Non-ionic Surfactants;” U.S. Pat. No. 5,328,404 for “Method of X-Ray Imaging Using lodinated Aromatic Propanedioates;” U.S. Pat. NO. 5,336,507 for “Use of Charged Phospholipids to Reduce Nanoparticle Aggregation;” U.S. Pat. No. 5,340,564 for “Formulations Comprising Olin 10-G to Prevent Particle Aggregation and Increase Stability;” U.S. Pat. No. 5,346,702 for “Use of Non-Ionic Cloud Point Modifiers to Minimize Nanoparticulate Aggregation During Sterilization;” U.S. Pat. No. 5,349,957 for “Preparation and Magnetic Properties of Very Small Magnetic-Dextran Particles;” U.S. Pat. No. 5,352,459 for “Use of Purified Surface Modifiers to Prevent Particle Aggregation During Sterilization;” U.S. Pat. Nos. 5,399,363 and 5,494,683, both for “Surface Modified Anticancer Nanoparticles;'U.S. Pat. No. 5,401,492 for “Water Insoluble Non-Magnetic Manganese Particles as Magnetic Resonance Enhancement Agents;” U.S. Pat. No. 5,429,824 for “Use of Tyloxapol as a Nanoparticulate Stabilizer;” U.S. Pat. No. 5,447,710 for “Method for Making Nanoparticulate X-Ray Blood Pool Contrast Agents Using High Molecular Weight Non-ionic Surfactants;” U.S. Pat. No. 5,451,393 for “X-Ray Contrast Compositions Useful in Medical Imaging;” U.S. Pat. No. 5,466,440 for “Formulations of Oral Gastrointestinal Diagnostic X-Ray Contrast Agents in Combination with Pharmaceutically Acceptable Clays;” U.S. Pat. No. 5,470,583 for “Method of Preparing Nanoparticle Compositions Containing Charged Phospholipids to Reduce Aggregation;” U.S. Pat. No. 5,472,683 for “Nanoparticulate Diagnostic Mixed Carbamic Anhydrides as X-Ray Contrast Agents for Blood Pool and Lymphatic System Imaging;” U.S. Pat. No. 5,500,204 for “Nanoparticulate Diagnostic Dimers as X-Ray Contrast Agents for Blood Pool and Lymphatic System Imaging;” U.S. Pat. No. 5,518,738 for “Nanoparticulate NSAID Formulations;” U.S. Pat. No. 5,521,218 for “Nanoparticulate lododipamide Derivatives for Use as X-Ray Contrast Agents;” U.S. Pat. No. 5,525,328 for “Nanoparticulate Diagnostic Diatrizoxy Ester X-Ray Contrast Agents for Blood Pool and Lymphatic System Imaging;” U.S. Pat. No. 5,543,133 for “Process of Preparing X-Ray Contrast Compositions Containing Nanoparticles;” U.S. Pat. No. 5,552,160 for “Surface Modified NSAID Nanoparticles;” U.S. Pat. No. 5,560,931 for “Formulations of Compounds as Nanoparticulate Dispersions in Digestible Oils or Fatty Acids;” U.S. Pat. No. 5,565,188 for “Polyalkylene Block Copolymers as Surface Modifiers for Nanoparticles;” U.S. Pat. No. 5,569,448 for “Sulfated Non-ionic Block Copolymer Surfactant as Stabilizer Coatings for Nanoparticle Compositions;” U.S. Pat. No. 5,571,536 for “Formulations of Compounds as Nanoparticulate Dispersions in Digestible Oils or Fatty Acids;” U.S. Pat. No. 5,573,749 for “Nanoparticulate Diagnostic Mixed Carboxylic Anydrides as X-Ray Contrast Agents for Blood Pool and Lymphatic System Imaging;” U.S. Pat. No. 5,573,750 for “Diagnostic Imaging X-Ray Contrast Agents;” U.S. Pat. No. 5,573,783 for “Redispersible Nanoparticulate Film Matrices With Protective Overcoats;” U.S. Pat. No. 5,580,579 for “Site-specific Adhesion Within the GI Tract Using Nanoparticles Stabilized by High Molecular Weight, Linear Poly(ethylene Oxide) Polymers;” U.S. Pat. No. 5,585,108 for “Formulations of Oral Gastrointestinal Therapeutic Agents in Combination with Pharmaceutically Acceptable Clays;” U.S. Pat. No. 5,587,143 for “Butylene Oxide-Ethylene Oxide Block Copolymers Surfactants as Stabilizer Coatings for Nanoparticulate Compositions;” U.S. Pat. No. 5,591,456 for “Milled Naproxen with Hydroxypropyl Cellulose as Dispersion Stabilizer;” U.S. Pat. No. 5,593,657 for “Novel Barium Salt Formulations Stabilized by Non-ionic and Anionic Stabilizers;” U.S. Pat. No. 5,622,938 for “Sugar Based Surfactant for Nanocrystals;” U.S. Pat. No. 5,628,981 for “Improved Formulations of Oral Gastrointestinal Diagnostic X-Ray Contrast Agents and Oral Gastrointestinal Therapeutic Agents;” U.S. Pat. No. 5,643,552 for “Nanoparticulate Diagnostic Mixed Carbonic Anhydrides as X-Ray Contrast Agents for Blood Pool and Lymphatic System Imaging;” U.S. Pat. No. 5,718,388 for “Continuous Method of Grinding Pharmaceutical Substances;” U.S. Pat. No. 5,718,919 for “Nanoparticles Containing the R(−)Enantiomer of Ibuprofen;” 5,747,001 for “Aerosols Containing Beclomethasone Nanoparticle Dispersions;” U.S. Pat. No. 5,834,025 for “Reduction of Intravenously Administered Nanoparticulate Formulation Induced Adverse Physiological Reactions;” U.S. Pat. No. 6,045,829 “Nanocrystalline Formulations of Human Immunodeficiency Virus (HIV) Protease Inhibitors Using Cellulosic Surface Stabilizers;” U.S. Pat. No. 6,068,858 for “Methods of Making Nanocrystalline Formulations of Human Immunodeficiency Virus (HIV) Protease Inhibitors Using Cellulosic Surface Stabilizers;” U.S. Pat. No. 6,153,225 for “Injectable Formulations of Nanoparticulate Naproxen;” U.S. Pat. No. 6,165,506 for “New Solid Dose Form of Nanoparticulate Naproxen;” U.S. Pat. No. 6,221,400 for “Methods of Treating Mammals Using Nanocrystalline Formulations of Human Immunodeficiency Virus (HIV) Protease Inhibitors;” U.S. Pat. No. 6,264,922 for “Nebulized Aerosols Containing Nanoparticle Dispersions;” U.S. Pat. No. 6,267,989 for “Methods for Preventing Crystal Growth and Particle Aggregation in Nanoparticle Compositions;” U.S. Pat. No. 6,270,806 for “Use of PEG-Derivatized Lipids as Surface Stabilizers for Nanoparticulate Compositions;” U.S. Pat. No. 6,316,029 for “Rapidly Disintegrating Solid Oral Dosage Form,” U.S. Pat. No. 6,375,986 for “Solid Dose Nanoparticulate Compositions Comprising a Synergistic Combination of a Polymeric Surface Stabilizer and Dioctyl Sodium Sulfosuccinate;” U.S. Pat. No. 6,428,814 for “Bioadhesive Nanoparticulate Compositions Having Cationic Surface Stabilizers;” and U.S. Pat. No. 6,432,381 for “Methods for Targeting Drug Delivery to the Upper and/or Lower Gastrointestinal Tract,” all of which are specifically incorporated by reference. In addition, U.S. Patent Application No. 20020012675 A1, published on Jan. 31, 2002, for “Controlled Release Nanoparticulate Compositions,” describes nanoparticulate compositions, and is specifically incorporated by reference.

Nanoparticulate peptides and proteins are referenced in U.S. Pat. No. 6,375,986 for “Solid Dose Nanoparticulate Compositions Comprising a Synergistic Combination of a Polymeric Surface Stabilizer and Dioctyl Sodium Sulfosuccinate,” U.S. Pat. No. 6,428,814 for “Bioadhesive Nanoparticulate Compositions Having Cationic Surface Stabilizers,” U.S. Patent Application No. 20020012675 A1 for “Controlled Release Nanoparticulate Compositions,” WO 00/51572 for “Use of PEG-Derivatized Lipids as Surface Stabilizers for Nanoparticle Compositions,” published on Sep. 8, 2000, and WO 00/53164 for “Methods for Preventing Crystal Growth and Particle Aggregation in Nanoparticulate Compositions, published on Sep. 14, 2000. However, none of these references teach or suggest that the peptide or protein can be insulin.

Amorphous small particle compositions are described, for example, in U.S. Pat. No. 4,783,484 for “Particulate Composition and Use Thereof as Antimicrobial Agent;” U.S. Pat. No. 4,826,689 for “Method for Making Uniformly Sized Particles from Water-Insoluble Organic Compounds;” U.S. Pat. No. 4,997,454 for “Method for Making Uniformly-Sized Particles From Insoluble Compounds;” U.S. Pat. No. 5,741,522 for “Ultrasmall, Non-aggregated Porous Particles of Uniform Size for Entrapping Gas Bubbles Within and Methods;” and U.S. Pat. No. 5,776,496, for “Ultrasmall Porous Particles for Enhancing Ultrasound Back Scatter.” None of these references relates to a nanoparticulate insulin composition.

B. Background Regarding Delivery of Peptides and Proteins

Delivery of peptides and proteins has long been a problem because of high toxicity, poor bioavailability, and degrading of the peptide or protein when formulated for administration or application. Numerous approaches to formulating peptide and protein compositions have been developed. For example, U.S. Pat. No. 5,889,110 discloses microparticles comprising a salt formed from a cation derived from a peptide containing one or more basic groups and an anion derived from a carboxy-terminated polyester. The composition is prepared from a stoichiometric equivalent of the polyester carboxylic acid end groups relative to the basic peptide groups, obtainable by a complicated and expensive process.

Such a process comprises: (i) dissolving the basic peptide and carboxy-terminated polyester in a first solvent in which both the peptide and the polyester are soluble to form a first solution; (ii) freezing the first solution at high speed to form a frozen mixture; (iii) freeze-drying the frozen mixture to remove the first solvent to form a freeze-dried product; (iv) dispersing the freeze-dried product into a second solvent which is a solvent for the polyester and a non-solvent for the peptide to form a second solution containing the peptide/polyester salt; and (v) removing the second solvent from the second solution by spray-drying, spray-congealing, evaporation, or phase separation coacervation to form a microparticulate solid product.

U.S. Pat. No. 5,354,562 discloses a process for preparing micronized polypeptide drugs in a powder form suitable for aerosol administration or for use in injectable suspensions by lyophilization followed by jet milling. U.S. Pat. No. 6,051,694 discloses a method of size reduction of proteins comprising contacting the solid protein with a critical fluid in which the protein is essentially insoluble, followed by depressurization of the protein and critical fluid mixture.

None of the prior art methods provide nanoparticulate insulin compositions. In fact, attempts to mill peptides such as insulin have resulted in a change of conformation of the peptides. For example, Leung et al., J. Pharm. Sci.,87:501-7 (1998), disclose transformation of the crystal structure of aspartame hemihydrate into a different polymorph upon ball milling of the peptide. This is not surprising, as peptides and proteins can be labile to milling conditions, e.g., shear, pressure, and temperature.

There remains a need in the art for nanoparticulate insulin formulations that would facilitate insulin delivery in high dosages with consistent and effective activity and other advantages in comparison to any conventional preparations presently available. In accordance with the present invention, such a preparation of insulin is provided.

### SUMMARY OF THE INVENTION

The present invention is directed to the surprising and unexpected discovery that stable nanoparticulate compositions of insulin can be made. The nanoparticulate compositions, which comprise insulin and at least one surface stabilizer adsorbed to the surface thereof, are advantageous in comparison to prior art insulin preparations in that they possess both a rapid onset of activity and prolonged activity.

The invention is further directed to methods of making nanoparticle insulin compositions and pharmaceutical dosage forms containing them. The method of preparing the insulin compositions comprises contacting insulin, e.g. insoluble bovine insulin or recombinant insulin, with at least one surface stabilizer for a time and under conditions sufficient to provide a stable nanoparticulate composition. The particle size of insulin is reduced by milling or homogenization. The one or more surface stabilizers can be contacted with the insulin either before, during, or after size reduction thereof.

The present invention is further directed to treatment of conditions requiring insulin therapy.

### BRIEF DESCRIPTION OF THE FIGURES

FIG. 1: Are scanning electron microscope images at 5,000 magnification of insulin particles before and after milling in accordance with the method of the present invention;

FIG. 2: Shows the results of a reducing SDS PAGE gel of the supernatant and pellet of a nanoparticulate insulin sample in two concentrations to determine whether milling caused a loss of insulin;

FIG. 3: Shows the results of a reducing SDS PAGE gel of the pellet of a nanoparticulate insulin sample in two concentrations to determine whether milling caused a loss of insulin;

FIG. 4: Shows the pharmacokinetic parameters of insulin resulting from injection of nanoparticulate insulin samples via intramuscular, subcutaneous and intraperitoneal routes in comparison to a control insulin solution:

FIG. 5: Is a bar graph illustrating the AUC (μU/ml.h) of insulin from injection of nanoparticulate insulin samples via subcutaneous, intramuscular and intraperitoneal routes in comparison to a control insulin solution;

FIG. 6: Is a plot showing blood insulin concentration over time following injection of nanoparticulate insulin samples via subcutaneous, intramuscular and intraperitoneal routes in comparison to a reference insulin solution;

FIG. 7: Is a plot showing blood glucose concentration over time following injection of nanoparticulate insulin samples via subcutaneous, intramuscular and intraperitoneal routes in comparison to a reference insulin solution; and

FIG. 8 Is a plot showing the change in blood glucose concentration over time following injection of nanoparticulate insulin samples via subcutaneous, intramuscular and intraperitoneal routes in comparison to a reference insulin solution.

### DETAILED DESCRIPTION OF THE INVENTION

The present invention is directed to compositions of nanoparticulate insulin and methods for their preparation and use in insulin therapy. Prior to the present invention, it was known that crystalline drugs could be formulated into nanoparticulate compositions by the method taught in the '684 patent. However, as discussed above, formulations of nanoparticulate insulin were not contemplated, partly due to the difficulty in retaining the insulin conformational structure upon milling to a particle size in the nanometer region. This is significant, as a change in conformation of insulin can correlate with a loss of activity.

Nanoparticulate insulin, prepared in accordance to the present invention, comprises at least one surface stabilizer that is adsorbed to the insulin particles. The surface stabilizer acts as a steric barrier to other insulin particles thereby preventing agglomeration and particle size growth. This results in a stable nanoparticulate composition, in which the particle size of the composition does not significantly increase over time via solubilization and recrystallization or agglomeration. Individually adsorbed molecules of the surface stabilizer are essentially free of intermolecular crosslinkages.

Insulin is a 5.8 kD protein hormone important in regulation of fuel metabolism. Secretion of insulin by the P cells of the pancreas is stimulated by glucose and the parasympathetic nervous system. Insulin promotes the dephosphorylation of key interconvertible enzymes. One consequence is to stimulate glycogen synthesis in both muscle and liver and to suppress gluconeogenesis by the liver. Insulin also accelerates glycolysis in the liver, which in turn increases the synthesis of fatty acids. The entry of glucose into muscle and adipose cells is promoted by insulin. The abundance of fatty acids and glucose in adipose tissue results in the synthesis and storage of triacylglycerols. The action of insulin also extends to amino acid and protein metabolism. Insulin promotes the uptake of branched-chain amino acids by muscle, which favors a building up of muscle protein. In sum, insulin has a general stimulating effect on protein synthesis.

Three different compositions of insulin are available: soluble, insoluble, and a mixture thereof. The difference among these compositions is in the observed bioavailability at physiological conditions. Insoluble insulin, and a mixture of soluble and insoluble insulin, have a slow onset of activity, about 2 hours, but a prolonged duration that extends for up to about 24 and 13-14 hours, respectively. Soluble insulin has a more rapid onset of activity, about 30 minutes, but with only about six hours duration of activity. The nanoparticle insulin of the present invention is superior to prior art insulin compositions in that it displays a rapid onset of activity, such as that found with soluble insulin, and it exhibits a prolonged duration of activity, such as that found in insoluble and soluble-insoluble insulin mixtures.

The insulin composition can be formulated for administration via, for example, oral, pulmonary, nasal, parenteral, rectal, local, buccal, or topical administration.

The insulin particles can be in the form of crystalline particles, semi-crystalline particles, semi-amorphous particles, amorphous particles, or a mixture thereof.

The concentration of insulin in the compositions of the invention can vary from about 99.5% to about 0.001%, from about 95% to about 0.1%, or from about 90% to about 0.5%, by weight, based on the total combined dry weight of the insulin and at least one surface stabilizer, not including other excipients.

A. Useful Surface Stabilizers

Suitable surface stabilizers utilized in preparing the subject nanoparticle insulin are preferably selected from known organic and inorganic pharmaceutical excipients. Two or more surface stabilizers may be used in combination. The surface stabilizers protect insulin from degradation and potential protease cleavage in addition to preventing agglomeration and particle size growth as described above.

Such organic and inorganic pharmaceutical excipients include various polymers, low molecular weight oligomers, natural products, and surfactants. Preferred surface stabilizers include nonionic and ionic surfactants (e.g., cationic and anionic surfactants). Representative examples of surface stabilizers include cetyl pyridinium chloride, gelatin, lecithin (phosphatides), dextran, glycerol, cholesterol, tragacanth, stearic acid, its salts and its esters, cetomacrogol emulsifying wax, sorbitan esters, polyoxyethylene alkyl ethers, polyoxyethylene castor oil derivatives, polyoxyethylene sorbitan fatty acid esters, e.g., the commercially available Tweens® such as Tween 80® (ICI Specialty Chemicals); polyethylene glycols, e.g., Carbowaxs 3350® and 1450®, and Carbopol 934® (Union Carbide), dodecyl trimethyl ammonium bromide, polyoxyethylene stearates, colloidal silicon dioxide, sodium dodecylsulfate, carboxymethylcellulose calcium, hydroxypropyl celluloses (e.g., HPC, HPC-SL, and HPC-L), hydroxypropyl methylcellulose (HPMC), carboxymethylcellulose sodium, methylcellulose, hydroxyethylcellulose, hydroxypropylmethyl-cellulose phthalate, noncrystalline cellulose, magnesium aluminum silicate, triethanolamine, polyvinyl alcohol (PVA), polyvinylpyrrolidone (PVP), 4-(1,1,3,3-tetramethylbutyl)-phenol polymer with ethylene oxide and formaldehyde (also known as tyloxapol), poloxamers, e.g., Pluronics F68® and F108®, which are block copolymers of ethylene oxide and propylene oxide; poloxamines, e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)); a charged phospholipid such as dimyristoyl phophatidyl glycerol, dioctylsulfosuccinate (DOSS); Tetronic 1508® (T-1508) (BASF Corporation), dialkylesters of sodium sulfosuccinic acid (e.g., Aerosol OT®, which is a dioctyl ester of sodium sulfosuccinic acid (American Cyanamid)); sodium lauryl sulfate (SLS), such as Duponol P® (DuPont); Tritons X-200®, which is an alkyl aryl polyether sulfonate (Rohm and Haas); Crodestas F-110®, which is a mixture of sucrose stearate and sucrose distearate (Croda Inc.); p-isononylphenoxypoly-(glycidol), also known as Olin-lOG® or Surfactant 10-G® (Olin Chemicals, Stamford, Conn.); Crodestas SL-40® (Croda, Inc.); poly (2-methacryloxyethyltrimethylammonium bromide) (S1001); poly(N-vinylpyrrolidone/2-dimethylaminoethyl methacrylate) di methylsulphate quatemary (S1002); poly(2-methylacryloxyamidopropyltrimethylammonium chloride)(S 1004); sodium deoxycholic acid; vitamin E derivatized with polyethylene glycol (PEG), a phospholipid derivatized with PEG, cholesterol derivatized with PEG, a cholesterol derivative derivatized with PEG, vitamin A derivatized with PEG, benzalkonium chloride (BKC), centrimide (a quaternary ammonium compound), Span® 20 (sorbitan monolaurate), Plasdone® S630, (a random copolymer of polyvinylpyrrolidone (PVP) and vinyl acetate in a 60:40 ratio), hexyldecyltrimethylammonium bromide (HDMAB), triblock copolymers of the structure —(-PEO)—(-PBO-)—(-PEO-)—(known as B20-5000), poly (2-methacryloxyethyltrimethylammonium bromide) (S 1001), poly(N-vinylpyrrolidone/2-dimethylaminoethyl methacrylate) di methylsulphate quaternary (S1002), poly(2-methylacryloxyamidopropyltrimethylammonium chloride) (S1004), random copolymers of PVP and vinyl acetate, lysozyme, and the like.

Examples of useful cationic surface stabilizers include, but are not limited to, polymers, biopolymers, polysaccharides, cellulosics, alginates, phospholipids, and nonpolymeric compounds, such as zwitterionic stabilizers, poly-n-methylpyridinium, anthryul pyridinium chloride, cationic phospholipids, chitosan, polylysine, polyvinylimidazole, polybrene, polymethylmethacrylate trimethylammoniumbromide bromide (PMMTMABr), hexyldesyltrimethylammonium bromide (HDMAB), and polyvinylpyrrolidone-2-dimethylaminoethyl methacrylate dimethyl sulfate.

Other useful cationic stabilizers include, but are not limited to, cationic lipids, sulfonium, phosphonium, and quarternary ammonium compounds, such as stearyltrimethylammonium chloride, benzyl-di(2-chloroethyl)ethylammonium bromide, coconut trimethyl ammonium chloride or bromide, coconut methyl dihydroxyethyl ammonium chloride or bromide, decyl triethyl ammonium chloride, decyl dimethyl hydroxyethyl ammonium chloride or bromide, C₁₂₋₁₅dimethyl hydroxyethyl ammonium chloride or bromide, coconut dimethyl hydroxyethyl ammonium chloride or bromide, myristyl trimethyl ammonium methyl sulphate, lauryl dimethyl benzyl ammonium chloride or bromide, lauryl dimethyl (ethenoxy)₄ ammonium chloride or bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts and dialkyl-dimethylammonium salts, lauryl trimethyl ammonium chloride, ethoxylated alkyamidoalkyldialkylammonium salt and/or an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride and dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂, C₁₅, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride (ALIQUAT 336™), POLYQUAT 10™, tetrabutylammonium bromide, benzyl trimethylammonium bromide, choline esters (such as choline esters of fatty acids), benzalkonium chloride, stearalkonium chloride compounds (such as stearyltrimonium chloride and Di-stearyldimonium chloride), cetyl pyridinium bromide or chloride, halide salts of quaternized polyoxyethylalkylamines, MIRAPOL™ and ALKAQUAT™ (Alkaril Chemical Company), alkyl pyridinium salts; amines, such as alkylamines, dialkylamines, alkanolamines, polyethylenepolyamines, N,N-dialkylaminoalkyl acrylates, and vinyl pyridine, amine salts, such as lauryl amine acetate, stearyl amine acetate, alkylpyridinium salt, and alkylimidazolium salt, and amine oxides; imide azolinium salts; protonated quaternary acrylamides; methylated quaternary polymers, such as poly[diallyl dimethylammonium chloride] and poly-[N-methyl vinyl pyridinium chloride]; and cationic guar.

Such exemplary cationic surface stabilizers and other useful cationic surface stabilizers are described in J. Cross and E. Singer, Cationic Surfactants: Analytical and Biological Evaluation (Marcel Dekker, 1994); P. and D. Rubingh (Editor), Cationic Surfactants: Physical Chemistry (Marcel Dekker, 1991); and J. Richmond, Cationic Surfactants: Organic Chemistry, (Marcel Dekker, 1990).

Particularly preferred nonpolymeric primary stabilizers are any nonpolymeric compound, such benzalkonium chloride, a carbonium compound, a phosphonium compound, an oxonium compound, a halonium compound, a cationic organometallic compound, a quarternary phosphorous compound, a pyridinium compound, an anilinium compound, an ammonium compound, a hydroxylammonium compound, a primary ammonium compound, a secondary ammonium compound, a tertiary ammonium compound, and quarternary ammonium compounds of the formula NR₁R₂R₃R₄⁽⁺⁾. For compounds of the formula NR₁R₂R₃R₄⁽⁺⁾:

(i) none of R₁—R₄ are CH₃;

(ii) one of R₁—R₄ is CH₃;

(iii) three of R₁—R₄ are CH₃;

(iv) all of R₁—R₄ are CH₃;

(v) two of R₁—R₄ are CH₃, one of R₁—R₄ is C₆H₅CH₂, and one of R₁—R₄ is an alkyl chain of seven carbon atoms or less;

(vi) two of R₁—R₄ are CH₃, one of R₁—R₄ is C₆H₅CH₂, and one of R₁—R₄ is an alkyl chain of nineteen carbon atoms or more;

(vii) two of R₁—R₄ are CH₃ and one of R₁—R₄ is the group C₆H₅(CH₂)ₙ, where n>1;

(viii) two of R₁—R₄ are CH₃, one of R₁—R₄ is C₆H₅CH₂, and one of R₁—R₄ comprises at least one heteroatom;

(ix) two of R₁—R₄ are CH₃, one of R₁—R₄ is C₆H₅CH₂, and one of R₁—R₄ comprises at least one halogen;

(x) two of R₁—R₄ are CH₃, one of R₁—R₄ is C₆H₅CH₂, and one of R₁—R₄ comprises at least one cyclic fragment;

(xi) two of R₁—R₄ are CH₃ and one of R₁—R₄ is a phenyl ring; or

(xii) two of R₁—R₄ are CH₃ and two of R₁—R₄ are purely aliphatic fragments.

Such compounds include, but are not limited to, behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride(Quaternium-14), Quaternium-22, Quatemium-26, Quaternium-18 hectorite, dimethylaminoethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide.

Most of these surface stabilizers are known pharmaceutical excipients and are described in detail in the Handbook of Pharmaceutical Excipients, published jointly by the American Pharmaceutical Association and The Pharmaceutical Society of Great Britain (The Pharmaceutical Press, 2000), specifically incorporated by reference. The surface stabilizers are commercially available and/or can be prepared by techniques known in the art starting from known materials.

The concentration of the at least one surface stabilizer can vary from about 0.001 to about 99.5%, from about 0. 1% to about 95%, and from about 0.5% to about 90%, by weight, based on the total combined dry weight of the insulin and at least one surface stabilizer, not including other excipients.

B. Particle Size of the Insulin Particles

Preferably, the insulin nanoparticles of the invention have an effective average particle size of less than about 5 microns, less than about 4 microns, less than about 3 microns, less than about 2 microns, less than about 1500 nm, less than about 1 micron, less than about 800 nm, less than about 700 nm, less than about 600 nm, less than about 500 nm, less than about 400 nm, less than about 300 nm, less than about 200 nm, less than about 100 nm, less than about 75 nm, or less than about 50 nm, as measured by light-scattering methods or other methods accepted in the art. By “an effective average particle size of less than about 5 microns” it is meant that at least about 50% of the insulin particles have a weight average particle size of less than about 5 microns when measured by light scattering or other conventional techniques. In addition, in other embodiments of the invention, at least about 70%, about 90%, or about 95% of the insulin particles have an effective average particle size of less than about 5 microns, less than about 4 microns, less than about 3 microns, less than about 2 microns, less than about 1500 nm, less than about 1 micron, less than about 800 nm, less than about 700 nm, less than about 600 nm, less than about 500 nm, less than about 400 nm, less than about 300 nm, less than about 200 nm, less than about 100 n,, less than about 75 nm, or less than about 50 nm.

C. Methods of Making Nanoparticulate Insulin Compositions

Nanoparticulate active agent compositions can be made using, for example, milling, or homogenization techniques. Exemplary methods of making nanoparticulate compositions are described in U.S. Pat. No. 5,145,684. Methods of making nanoparticulate compositions are also described in U.S. Pat. No. 5,518,187 for “Method of Grinding Pharmaceutical Substances;” U.S. Pat. No. 5,718,388 for “Continuous Method of Grinding Pharmaceutical Substances;” U.S. Pat. No. 5,862,999 for “Method of Grinding Pharmaceutical Substances;” U.S. Pat. No. 5,543,133 for “Process of Preparing X-Ray Contrast Compositions Containing Nanoparticles;” U.S. Pat. No. 5,534,270 for “Method of Preparing Stable Drug Nanoparticles;” U.S. Pat. No. 5,510,118 for “Process of Preparing Therapeutic Compositions Containing Nanoparticles;” and U.S. Pat. No. 5,470,583 for “Method of Preparing Nanoparticle Compositions Containing Charged Phospholipids to Reduce Aggregation,” all of which are specifically incorporated by reference.

The optimal effective average particle size of the invention can be obtained by controlling the process of particle size reduction, such as by controlling the milling time and the amount of surface stabilizer added. Particle size growth and particle aggregation can also be minimized by milling and/or storing the composition under colder temperatures.

The nanoparticulate insulin dispersions made via, for example, milling or homogenization can be utilized in solid, semi-solid, or liquid dosage formulations, such as controlled release formulations, solid dose fast melt formulations, aerosol formulations, lyophilized formulations, tablets, solid lozenge, capsules, powders, liquids for injection, etc.

1. Milling to Obtain Nanoparticulate Insulin Compositions

Milling insulin to obtain a nanoparticulate composition comprises dispersing insulin particles in a liquid dispersion medium in which insulin is poorly soluble, followed by applying mechanical means in the presence of grinding media to reduce the particle size of the insulin to the desired effective average particle size. The dispersion medium can be, for example, water, safflower oil, ethanol, t-butanol, glycerin, polyethylene glycol (PEG), hexane, or glycol.

The insulin particles can be reduced in size in the presence of at least one surface stabilizer. Alternatively, the insulin particles can be contacted with one or more surface stabilizers after attrition. It is preferred, however, to disperse the insulin in the liquid dispersion medium in the presence of the at least one surface stabilizer as an aid in wetting of the insulin particles. Other compounds, such as a diluent, can be added to the insulin/surface stabilizer composition during the size reduction process. Dispersions can be manufactured continuously or in a batch mode.

2. Homogenization to Obtain Insulin Nanoparticulate Compositions

Exemplary homogenization methods of preparing active agent nanoparticulate compositions are described in U.S. Pat. No. 5,510,118, for “Process of Preparing Therapeutic Compositions Containing Nanoparticles.”

Such a method comprises dispersing insulin particles in a liquid dispersion medium, followed by subjecting the dispersion to homogenization to reduce the particle size of the insulin to the desired effective average particle size. The insulin particles can be reduced in size in the presence of at least one surface stabilizer. Alternatively, the insulin particles can be contacted with one or more surface stabilizers either before or after particle size reduction. It is preferred, however, to disperse the insulin in the liquid dispersion medium in the presence of the at least one surface stabilizer as an aid to wetting of the insulin particles. Other compounds, such as a diluent, can be added to the insulin/surface stabilizer composition either before, during, or after the size reduction process. Dispersions can be manufactured continuously or in a batch mode.

D. Excipients

Pharmaceutical compositions in accordance with the present invention include nanoparticulate insulin compositions formulated together with one or more non-toxic, physiologically acceptable carriers, adjuvants, or vehicles, collectively referred to as carriers. Such carriers may comprise physiologically acceptable sterile aqueous or nonaqueous solutions, dispersions, suspensions or emulsions, and sterile powders for reconstitution into such preparations. Examples of suitable aqueous and nonaqueous carriers, diluents, solvents, or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

The nanoparticulate compositions may also contain adjuvants such as preserving, wetting, emulsifying, and dispensing agents. Prevention of the growth of microorganisms can be ensured by various antibacterial and antifungal agents, such as potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quarternary compounds such as benzalkonium chloride, parabens, chlorobutanol, phenol, sorbic acid, and the like. It may also be desirable to include isotonic agents, such as sugars, sodium chloride, and the like. Prolonged absorption of the injectable pharmaceutical form can be brought about by the use of agents delaying absorption, such as aluminum monostearate and gelatin.

In one embodiment of the invention, the nanoparticulate compositions of the invention are intended for parenteral administration to humans and animals by known routes of parenteral administration, i.e., intravenous, intramuscular, subcutaneous, or intraperitoneal. Other dosage forms can also be utilized, such as aerosols, tablets, etc.

Actual dosage levels of the nanoparticulate insulin compositions of the invention are given as standardized units of insulin as is the case with conventional insulin preparations and the dosage administered will depend on recognized factors such as the time of administration and the body weight, general health, age, sex, and diet of the patient.

The following examples are given to illustrate the present invention. It should be understood, however, that the invention is not to be limited to the specific conditions or details described in these examples. Throughout the specification, all references to publicly available documents, including U.S. patents, are specifically incorporated herein by reference.

### EXAMPLE 1

The purpose of this example was to prepare nanoparticulate insulin formulations using low energy milling techniques.

Insulin and one or more surface stabilizers, in the amounts shown in Table 1, were mixed with water to form a pre-milling slurry. This slurry was then added to a sealable vessel and rotated for from 1 to 4 days at a pre-set rotational speed (typically 100-200 rpm) on a roller mill using high wear zirconia grinding media (Tosoh Corporation, Tokyo, Japan) with a diameter of 0.8 mm. This low energy milling technique relies upon gravitational grinding mechanisms to break the particle size down, hence the use of heavy ceramic media.

Particle size distributions of the resultant insulin compositions were determined using a Horiba LA-910 light-scattering particle size analyzer (Horiba Instruments, Irvine, Calif.). The results shown in Table 1 were determined at 24 hours post milling. It is to be noted that the concentration of insulin in this Example was 5%, but could have been, for example, 1%, 2%, 3%, or 10% or higher.

The surface stabilizers used include: (1) poly (2-methacryloxyethyl-trimethylammonium bromide) (S1001); (2) poly(N-vinylpyrrolidone/2-dimethyl-aminoethyl methacrylate) dimethylsulphate quarternary (S1002); (3) poly(2-methylacryloxyamido-propyltrimethylammonium chloride) (S1004); (4) Pluronic® F68; (5) hydroxypropyl cellulose (HPC); (6) sodium deoxycholic acid; (7) vitamin E derivatized with polyethylene glycol (PEG); and (8) Tween® 80.

TABLE 1
Low Energy Ball-Milled Nanoparticulate Insulin Formulations
(Particle Size 24 hrs after Milling)

| | | | Mean Particle | Particle size |
|---|---|---|---|---|
| | | | Size @ 24 | of 90% @ 24 |
| Exp | % | % of | hours after | hours after |
| No. | Drug | Stabilizer | milling (nm) | milling (nm) |
| 1 | 5.0% | 0.0 | 4198 (1344 | 7092 (3104 |
| | insulin | | after 1 min | after 1 min |
| | | | sonication) | sonication) |
| 2 | 5.0% | 1% F68 0.1% | 165 (144 | 266 (193 |
| | insulin | Sodium | after 1 min | after 1 min |
| | | Deoxycholic | sonication) | sonication) |
| | | Acid | | |
| 3 | 5.0% | 1% PVP k17 | 199 (163 | 279 (219 |
| | insulin | 0.1% Sodium | after 1 min | after 1 min |
| | | Deoxycholic | sonication) | sonication) |
| | | Acid | | |
| 4 | 5.0% | 1% PVP k29-32 | 243 (171 | 364 (250 |
| | insulin | 0.1% Sodium | after 1 min | after 1 min |
| | | Deoxycholic | sonication) | sonication) |
| | | Acid | | |
| 5 | 5.0% | 1% S1004 | 252 | 362 |
| | insulin | | | |
| 6 | 5.0% | 1% S1001 | 282 (258 | 401 (380 |
| | insulin | | after 1 min | after 1 min |
| | | | sonication) | sonication) |

The measurements reported in Table 2 were taken at one week post milling to determine stability for a representative group of the nanoparticulate compositions shown in Table 1. In several instances, the particle size of the composition was smaller after one week than at 24 hours post milling. While not being bound to any one theory, it is believed that the stabilizer and drug particle may go through a thermodynamic relaxation period. Another theory is that in conjunction with relaxation or separately therefrom, there are interactions between the insulin particle and the stabilizer. Further, a phenomenon may be occurring at the surface of the insulin particle that is similar to the use of cationic stabilizers to complex and condense DNA in solution, i.e. the stabilizer interaction may cause a slight condensation on the surface of the insulin particle, resulting in the observed reduction in particle size.

TABLE 2
Low Energy Ball-Milled Nanoparticulate Insulin Formulations
(Particle Size 24 hrs and 1 Week After Milling)

| | | | Mean | Particle size | Mean | Particle size |
|---|---|---|---|---|---|---|
| | | | Particle Size | of 90% | Particle Size | of 90% |
| | | | @ 24 hours | @ 24 hours | @ 1 week | @ 1 week |
| Exp | % | % of | after milling | after milling | after milling | after milling |
| No. | Drug | Stabilizer | (nm) | (nm) | (nm) | (nm) |
| 4 | 5.0% | 1% PVP k29-32 | 243 | 364 | 212 | 301 |
| | insulin | 0.1% Sodium | (171 after | (250 after | (165 after | (222 after |
| | | Deoxycholic | 1 min | 1 min | 1 min | 1 min |
| | | Acid | sonication) | sonication) | sonication) | sonication) |
| 5 | 5.0% | 1% S1004 | 252 | 362 | 263 | 364 |
| | insulin | | | | | |
| 6 | 5.0% | 1% S1001 | 282 | 401 | 303 | 450 |
| | insulin | | (258 after | (380 after | (266 after | (384 after |
| | | | 1 min | 1 min | 1 min | 1 min |
| | | | sonication) | sonication) | sonication) | sonication) |
| 7 | 5.0% | 1% S1001 | 521 | 1004 | 498 | 905 |
| | insulin | | | | | |

### EXAMPLE 2

The purpose of this example was to prepare nanoparticulate insulin formulations using high energy milling conditions.

Samples of insulin were prepared using a high energy attrition media mill, NanoMil™ (Elan Drug Delivery, King of Prussia, Pa.). A high energy mill is designed to apply a much higher rotational velocity to the particulate dispersion (100-6000 rpm, typically 5000 rpm), than that used in low energy milling processes. The elevated rotational velocity imparts very high shear conditions within the milling chamber. It is this shear force which causes the particle size reduction.

The media used in this milling technology is a much lighter, highly crosslinked polystyrene media. For preparation of nanoparticulate insulin compositions described in Table 3, 500 μm media was used. Other media sizes that could be used range from 50 μm to 500 μm. The samples were milled from about 30 minutes up to about 3 hours. Particle size distributions were determined using a Horiba LA-910 light-scattering particle size analyzer (Horiba Instruments, Irvine, Calif.). Particle size was determined for all three compositions following milling, for Sample 2 at 24 hours following milling, and for Samples 1 and 2 at 1 week after milling. The results are shown in Table 3.

TABLE 3
HIGH ENERGY MILLED NANOPARTICULATE INSULIN FORMULATIONS

| | | | | | Mean | Particle | Mean | Particle |
|---|---|---|---|---|---|---|---|---|
| | | | Mean | Particle | Particle | Size of | Particle | size of |
| | | | Particle | Size (nm) | Size (nm) | 90% (nm) | Size (nm) | 90% (nm) |
| Exp. | % of | % of | Size (nm) | of 90% | @ 24 hrs | @ 24 hrs | @ 1 week | @ 1 week |
| No. | Insulin | Stabilizer | @ harvest | @ harvest | after milling | after milling | after milling | after milling |
| 1 | 4.0% | 1% S1001 | 255 | 378 | | | 297 | 436 |
| | | | 234 | 353 | | | 247 | 372 |
| 2 | 2.5% | 1% S1004 | 282 | 381 | 284 | 382 | 232 | 329 |
| | | | 239 | 341 | 275 | 370 | 217 | 207 |

These results demonstrate the ability to produce nanoparticulate insulin compositions which maintain stability and perhaps even improve upon storage using high energy milling techniques. Most importantly, the results demonstrate that, upon storage, little or no aggregation is observed. Thus, the data show the ability to consistently prepare nanoparticulate insulin formulations which maintain their integrity upon storage.

### EXAMPLE 3

The purpose of this example is to examine nanoparticle insulin prepared in accordance with the invention and test the stability of the compositions over an extended storage period.

Nanoparticulate insulin was prepared in accordance with the procedure of Example 2. Milling was carried out until the insulin particles were found to have a mean particle size of 100 nm, with 90% of the particles less than 145 nm.

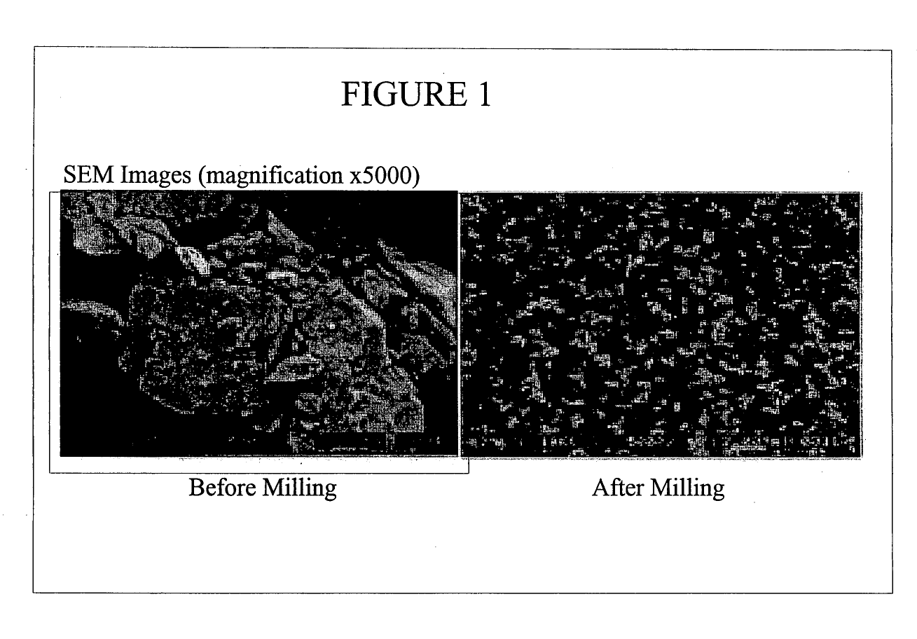

FIG. 1 shows SEM images of insulin particles before and after milling, clearly demonstrating the effect of the milling and, more importantly, the capacity of the surface stabilizers to prevent agglomeration of the insulin particles. Analysis of a sample of this material stored for six months at 5° C. revealed 90% of the insulin particles to be smaller than 145 nm.

### EXAMPLE 4

The purpose of this example was to determine the insulin content of a nanoparticulate composition following milling, and to determine if the milling process results in loss of insulin.

Nanoparticulate insulin was prepared in accordance with the procedure of Example 1 from a slurry of 2% insulin having 1% Pluronic® F68 and 0.1% sodium deoxycholic acid as surface stabilizers.

Aliquots of the nanoparticulate insulin were centrifuged at 13,000 g for 15 min. The supernatant was isolated and the pellet washed in Phosphate Buffer Saline (PBS) and re-spun. The pellets and supernatant fractions post-centrifugation were subjected to analysis on an SDS-PAGE gel. Raw insulin in PBS additionally containing 0.1 M HCl for stability (Sigma 1-5500) served as a control.

Reducing gels used in the analysis were prepared as 16% acrylamide gels and comprised 5.33 ml of acrylamide solution, 2.02 ml distilled water, 2.50 ml of 1.5 M TRIS buffer at pH of 8.8, 100 μm 10% SDS, 50 μm 10% ammonium persulfate, and 5 μm of TEMED (N,N,N′,N′-tetramethylethylene diamine). Reducing gels are useful for visualizing insulin bands, which appear as a dimer, tetramer, etc., and for identifying degraded insulin fragments.

The composition of the running buffer and loading buffers were as follows:

| 5× Electrode Buffer | | | Sample Loading Buffer | |
|---|---|---|---|---|
| (1× = 25 mM Tris, | | | (SDS reducing buffer: 62.5 mM | |
| 192 mM Glycine, 0.1% SDS, | | | Tris HCl (pH 6.8), 20% Glycerol, | |
| pH 8.3) | | | 2% SDS, 5% β-mercaptoethanol) | |
| Tris Base | 37.5 | g | Distilled water | 3.0 ml |
| Glycine | 180.0 | g | 0.5M Tris-HCl (pH 6.8) | 1.0 ml |
| SDS | 12.5 | g | Glycerol | 1.6 ml |
| Distilled Water | 2.5 | L | 10% SDS | 1.6 ml |

| | | |
|---|---|---|
| Dilute 200 ml of the 5× | β-mercaptoethanol | 0.4 ml |
| stock with 800 ml distilled | 0.5% (w/v) bromophenol | 0.4 ml |
| water before use. | blue (in water) | |

The samples and the supernatant were diluted to 1:10 and 1:50, respectively, with sample buffer and then heated to 95° C. for 5 mins before loading onto the gel. The volume of supernatant loaded was the amount that would be needed to load the required amount of sample, if the insulin were left in suspension.

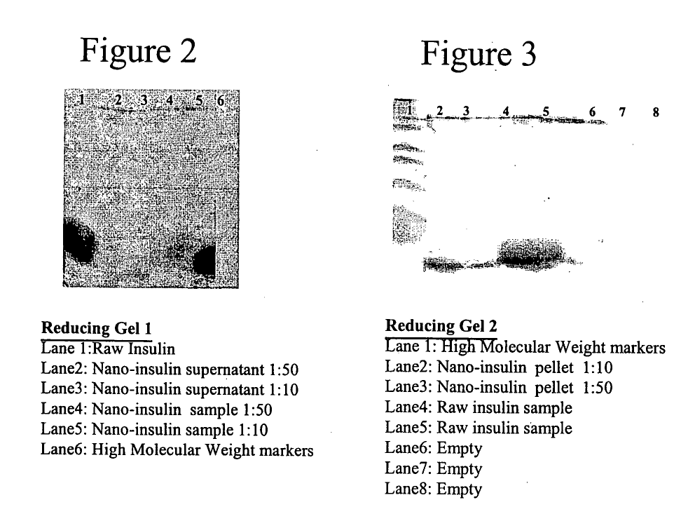

Two reducing gels were prepared, and the samples were loaded onto the gels so that the pellet and supernatant fractions from each sample could be observed side by side. The results are shown in FIG. 2 and FIG. 3. The reducing gel shown in FIG. 2 was loaded with the following:

Lane 1: Raw Insulin Sample

Lane 2: Sample supernatant 1:50 dilution;

Lane 3: Sample supernatant 1:10 dilution;

Lane 4: Pellet Sample 1:50 dilution;

Lane 5: Pellet Sample 1:10 dilution; and

Lane 6: High Molecular Weight markers.

The reducing gel shown in FIG. 3 was loaded with the following:

Lane 1: High Molecular Weight markers;

Lane 2: Pellet Sample 1:10 dilution;

Lane 3: Pellet Sample 1:50 dilution;

Lane 4: Raw Insulin Sample; and

Lane 5: Raw Insulin Sample.

The gels shown in FIGS. 2 and 3 demonstrate that the pellet fraction of the milled insulin formulation contains all of the insulin. The lanes loaded with supernatant appear to be free of insulin. This indicates that all of the insulin in the nanoparticulate insulin composition is entrapped within or associated with the nanoparticulate composition, with little or no insulin free in solution. Thus, there is essentially 100% entrapment or association of the insulin within the nanoparticulate composition.

### EXAMPLE 5

The purpose of this example was to test the effectiveness of nanoparticulate insulin compositions parenterally administered, and to compare the effectiveness of the formulations with conventional raw insulin compositions.

All animals (Wistar rats) were weighed and fasted for 4 hours prior to study initiation. The rats (250-300 g) were anaesthetized by intramuscular (IM) administration of a solution of ketamine (22.5 mg/animal) and acepromazine (0.75 mg/kg) one hour prior to administration of the test solutions. The sample of nanoparticulate insulin prepared in accordance with the procedure of Example 1 from a slurry of 2% insulin having 1% Pluronic® F68 and 0.1% sodium deoxycholic acid as surface stabilizers was formulated as a solution in PBS (phosphate buffer solution) pH 7.4. Groups of 6 animals received an amount of the solution sufficient to provide one IU of insulin by subcutaneous, intramuscular, or intraperitoneal injection in a randomized non-crossover study. Blood glucose values for the rats were measured using an Accutren® alpha glucometer (Boehringer Mannhein) following systemic blood samples that were taken from the tail artery. A baseline blood sample was taken and samples were taken at intervals post injection up to four hours. Anesthesia was maintained throughout the study. A non-formulated insulin solution was utilized as a reference.

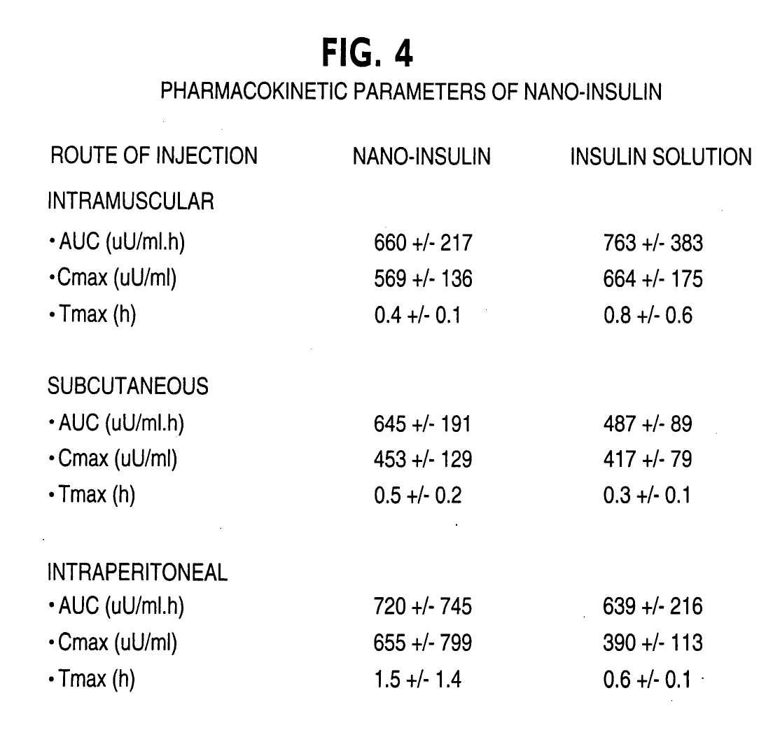

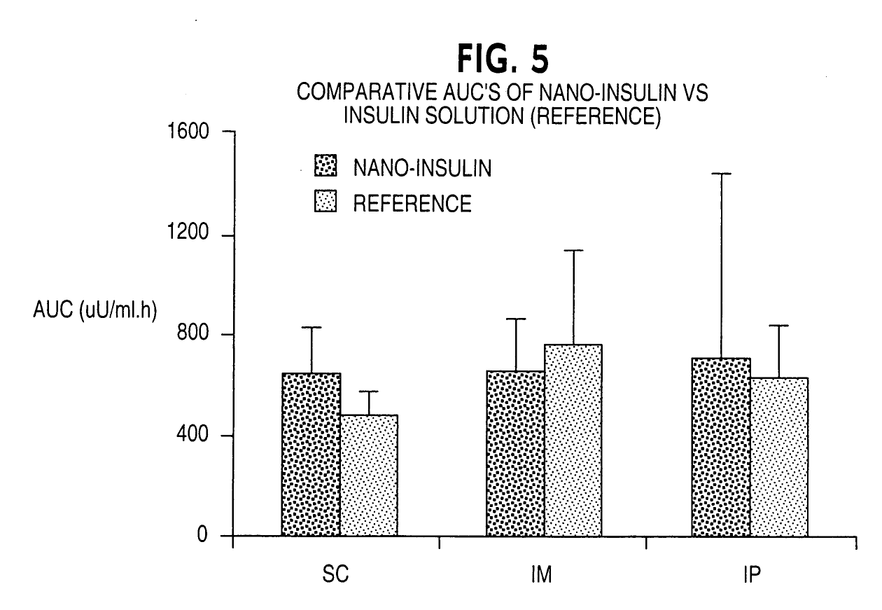

The rat model used is a hypergylcemic model. The pharmacokinetic parameters of the test are given in FIG. 4. It can be seen from the results in FIG. 4 that the insulin compositions of the present invention are biologically active and at least approximately equal to the reference insulin solution. FIG. 5 illustrates the AUC (μU/ml.h) for the sample by the three modes of parenteral administration in comparison to the reference insulin solution. The analysis shown in FIG. 5 likewise establishes the equivalence of the test preparation to the reference insulin solution.

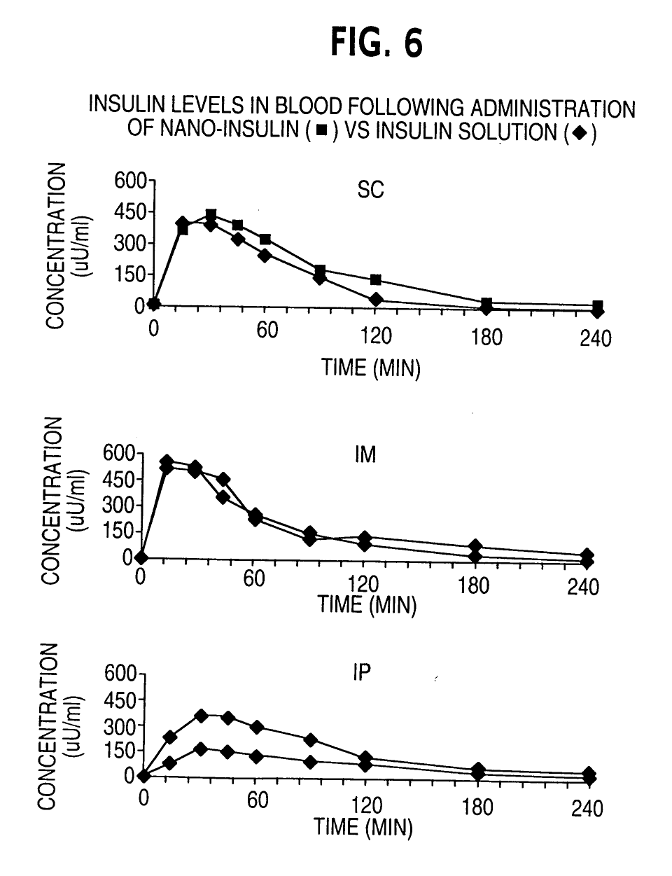

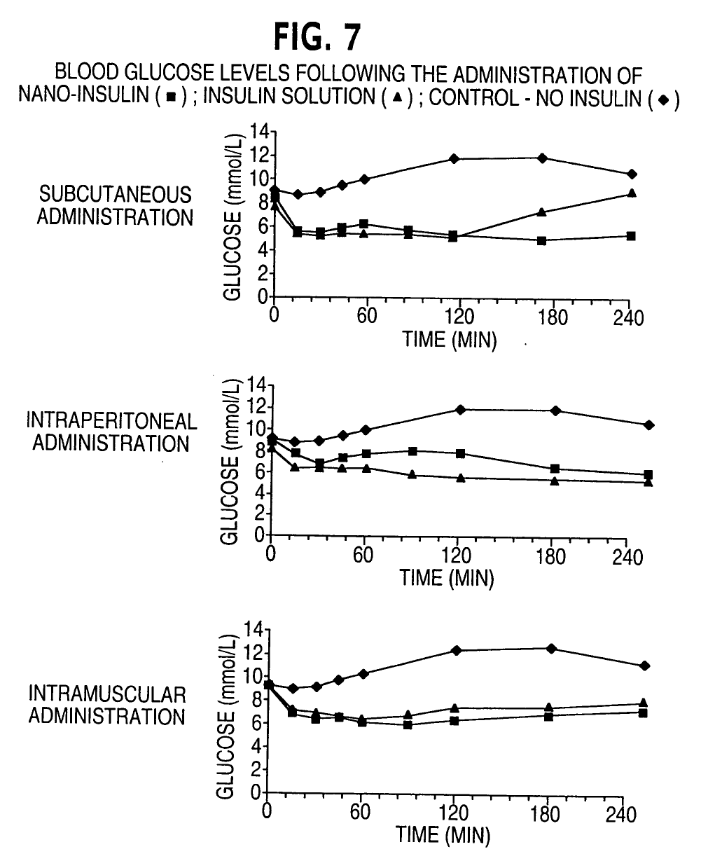

FIGS. 6 and 7 are companion concentration-time profiles in that FIG. 6 illustrates mean glucose concentration while FIG. 7 illustrates mean insulin concentration. As described above, both plot the test sample administered via subcutaneous, intramuscular and intraperitoneal injection in comparison to a reference insulin preparation. If an insulin preparation is biologically active, a fall in blood glucose levels is expected to coincide with the rise in blood insulin levels. The profiles in FIGS. 6 and 7 clearly establish that the nanoparticulate insulin formulations of the invention are biologically active.

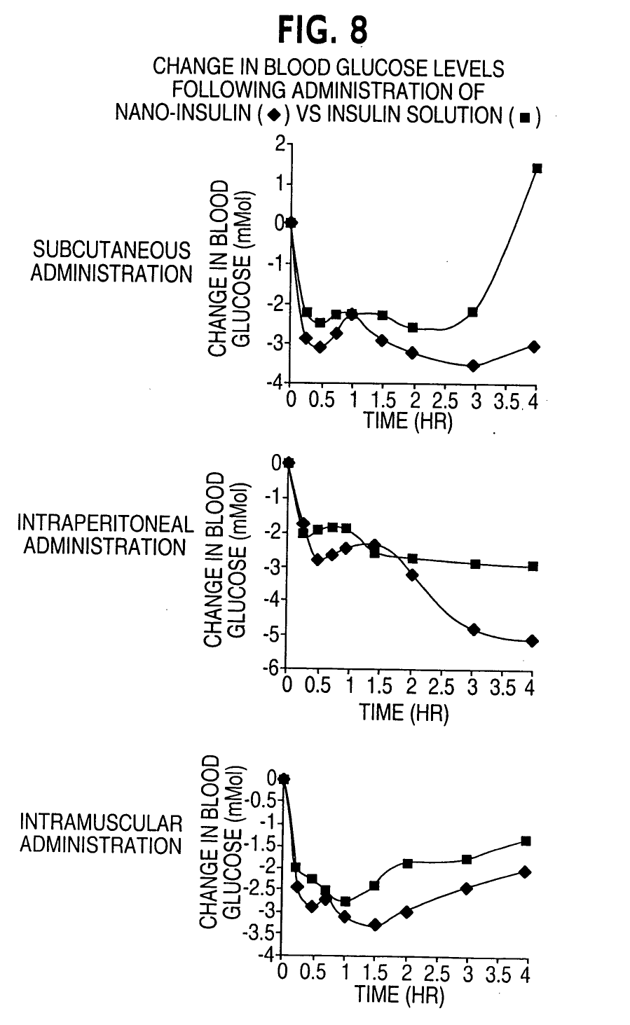

Overall, the results reported in the figures demonstrate that the nanoparticulate insulin compositions of the present invention have excellent bioactivity following parenteral administration, which is at least equivalent to the reference insulin solution tested. In the plot of change in blood glucose level plotted in FIG. 8, it can be seen that the nanoparticle preparation of the present invention is advantageous over the reference insulin solution in maintaining a lowering of the blood glucose level over the duration of the test. A particular advantage can be seen from the comparison via intraperitoneal administration.

It will be apparent to those skilled in the art that various modifications and variations can be made in the methods and compositions of the present invention without departing from the spirit or scope of the invention. Thus, it is intended that the present invention covers the modifications and variations of this invention provided they come within the scope of the appended claims and their equivalents. The preceding examples further illustrate the invention and are not to be construed as limiting of the specification and claims in any way.

We claim:

1. A nanoparticulate composition comprising insulin particles and at least one non-crosslinked surface stabilizer adsorbed on the surface thereof, wherein the insulin particles have an effective average particle size of less than about 5 microns.

2. The composition of claim 1, wherein the effective average particle size of the insulin particles is selected from the group consisting of less than about 4 microns, less than about 3 microns, less than about 2 microns, less than about 1500 nm, less than about 1 micron, less than about 800 nm, less than about 700 nm, less than about 600 nm, less than about 500 nm, less than about 400 nm, less than about 300 nm, less than about 200 nm, less than about 100 nm, less than about 75 nm, and less than about 50 nm.

3. The composition of claim 1, wherein the concentration of insulin is selected from the group consisting of from about 99.5% to about 0.001%, from about 95% to about 0.1%, and from about 90% to about 0.5%, by weight, based on the total combined dry weight of the insulin particles and at least one surface stabilizer, not including other excipients.

4. The composition of claim 1, wherein the concentration of the at least one surface stabilizer is selected from the group consisting of from about 0.001 to about 99.5%, from about 0.1 to about 95%, and from about 0.5 to about 90%, by weight, based on the total combined dry weight of the insulin particles and at least one surface stabilizer, not including other excipients.

5. The composition of claim 1, wherein the composition further comprises one or more pharmaceutically acceptable excipients, carriers, or a combination thereof.

6. The composition of claim 1, wherein the composition is formulated for administration selected from the group consisting of oral, pulmonary, nasal, parenteral, rectal, local, buccal, and topical administration.

7. A dosage form comprising the nanoparticulate insulin composition of claim 1, wherein the dosage form is a solid, semi-solid, or liquid dosage formulation.

8. A dosage form comprising the nanoparticulate insulin composition of claim 1, wherein the dosage form is selected from the group consisting of controlled release formulations, solid dose fast melt formulations, aerosol formulations, lyophilized formulations, tablets, solid lozenge, capsules, powders, and liquids for injection.

9. A pharmaceutical composition for parenteral administration comprising the nanoparticulate insulin composition of claim 1 and a suitable, pharmaceutically acceptable excipient.

10. The composition of claim 1, wherein the insulin is in a form selected from the group consisting of crystalline particles, semi-crystalline particles, semi-amorphous particles, amorphous particles, and a mixture thereof.

11. The composition of claim 1, further comprising at least two surface stabilizers.

12. The composition of claim 1, wherein the at least one surface stabilizer is selected from the group consisting of a nonionic surface stabilizer, an anionic surface stabilizer, a cationic surface stabilizer, and an ionic surface stabilizer.

13. The composition of claim 12, wherein the at least one surface stabilizer is selected from the group consisting of cetyl pyridinium chloride, gelatin, casein, phosphatides, dextran, glycerol, gum acacia, cholesterol, tragacanth, stearic acid, stearic acid esters and salts, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hydroxypropyl methylcellulose, carboxymethylcellulose sodium, methylcellulose, hydroxyethylcellulose, hydroxypropylmethyl-cellulose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers, poloxamines, a charged phospholipid, dimyristoyl phophatidyl glycerol, dioctylsulfosuccinate, dialkylesters of sodium sulfosuccinic acid, sodium lauryl sulfate, alkyl aryl polyether sulfonates, mixtures of sucrose stearate and sucrose distearate, triblock copolymers of the structure: -(-PEO)—(-PBO—)—(-PEO—)—, p-isononylphenoxypoly-(glycidol), decanoyl-N-methylglucamide; n-decyl β-D-glucopyranoside, n-decyl β-D-maltopyranoside, n-dodecyl β-D-glucopyranoside, n-dodecyl β-D-maltoside, heptanoyl-N-methylglucamide, n-heptyl-β-D-glucopyranoside, n-heptyl β-D-thioglucoside, n-hexyl β-D-glucopyranoside, nonanoyl-N-methylglucamide, n-noyl β-D-glucopyranoside, octanoyl-N-methylglucamide, n-octyl-β-D-glucopyranoside, octyl β-D-thioglucopyranoside, lysozyme, a PEG derivatized phospholipid, PEG derivatized cholesterol, a PEG derivatized cholesterol derivative, PEG derivatized vitamin A, PEG derivatized vitamin E, and random copolymers of vinyl acetate and vinyl pyrrolidone.

14. The composition of claim 12, wherein the at least one cationic surface stabilizer is selected from the group consisting of a polymer, a biopolymer, a polysaccharide, a cellulosic, an alginate, a nonpolymeric compound, and a phospholipid.

15. The composition of claim 12, wherein the at least one surface stabilizer is selected from the group consisting of cationic lipids, benzalkonium chloride, sulfonium compounds, phosphonium compounds, quarternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅dimethyl hydroxyethyl ammonium chloride, C₁₂₋₁₅dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl (ethenoxy)₄ ammonium chloride, lauryl dimethyl (ethenoxy)₄ ammonium bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, C₁₅ trimethyl ammonium bromides, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, POLYQUAT 10™, tetrabutylammonium bromide, benzyl trimethylammonium bromide, choline esters, benzalkonium chloride, stearalkonium chloride compounds, cetyl pyridinium bromide, cetyl pyridinium chloride, halide salts of quatemized polyoxyethylalkylamines, MIRAPOL™, ALKAQUAT™, alkyl pyridinium salts; amines, amine salts, amine oxides, imide azolinium salts, protonated quaternary acrylamides, methylated quaternary polymers, cationic guar, polymethylmethacrylate trimethylammonium bromide, polyvinylpyrrolidone-2-dimethylaminoethyl methacrylate dimethyl sulfate, hexadecyltrimethyl ammonium bromide, poly (2-methacryloxyethyltrimethylammonium bromide) (S1001), poly(N-vinylpyrrolidone/2-dimethylaminoethyl methacrylate) di methylsulphate quarternary (S1002), and poly(2-methylacryloxyamidopropyltrimethylammonium chloride) (S1004).

16. A method of making a nanoparticulate insulin composition comprising contacting insulin particles with at least one surface stabilizer for a time and under conditions sufficient to provide a nanoparticulate insulin composition having an effective average particle size of less than about 5 microns.

17. The method of claim 16, wherein said contacting comprises milling.

18. The method of claim 17, wherein said milling is high energy milling.

19. The method of claim 17, wherein said milling comprises wet milling.

20. The method of claim 16, wherein said contacting comprises homogenizing.

21. The method of claim 16, wherein the effective average particle size of the insulin particles is selected from the group consisting of less than about 4 microns, less than about 3 microns, less than about 2 microns, less than about 1500 nm, less than about 1 micron, less than about 800 nm, less than about 700 nm, less than about 600 nm, less than about 500 nm, less than about 400 nm, less than about 300 nm, less than about 200 nm, less than about 100 nm, less than about 75 nm, and less than about 50 nm.

22. The method of claim 16, wherein the concentration of insulin is selected from the group consisting of from about 99.5% to about 0.001%, from about 95% to about 0.1%, and from about 90% to about 0.5%, by weight, based on the total combined dry weight of the insulin particles and at least one surface stabilizer, not including other excipients.

23. The method of claim 16, wherein the concentration of the at least one surface stabilizer is selected from the group consisting of from about 0.001 to about 99.5%, from about 0.1 to about 95%, and from about 0.5 to about 90%, by weight, based on the total combined dry weight of the insulin particles and at least one surface stabilizer, not including other excipients.

24. The method of claim 16, wherein the insulin is in a form selected from the group consisting of crystalline particles, semi-crystalline particles, semi-amorphous particles, amorphous particles, and a mixture thereof.

25. The method of claim 16, wherein the composition further comprises at least two surface stabilizers.

26. The method of claim 16, wherein the at least one surface stabilizer is selected from the group consisting of a nonionic surface stabilizer, an anionic surface stabilizer, a cationic surface stabilizer, and an ionic surface stabilizer.

27. The method of claim 26, wherein the at least one surface stabilizer is selected from the group consisting of cetyl pyridinium chloride, gelatin, casein, phosphatides, dextran, glycerol, gum acacia, cholesterol, tragacanth, stearic acid, stearic acid esters and salts, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hydroxypropyl methylcellulose, carboxymethylcellulose sodium, methylcellulose, hydroxyethylcellulose, hydroxypropylmethyl-cellulose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers, poloxamines, a charged phospholipid, dimyristoyl phophatidyl glycerol, dioctylsulfosuccinate, dialkylesters of sodium sulfosuccinic acid, sodium lauryl sulfate, alkyl aryl polyether sulfonates, mixtures of sucrose stearate and sucrose distearate, triblock copolymers of the structure: -(-PEO)—(-PBO—)—(-PEO—)—, p-isononylphenoxypoly-(glycidol), decanoyl-N-methylglucamide; n-decyl β-D-glucopyranoside, n-decyl β-D-maltopyranoside, n-dodecyl β-D-glucopyranoside, n-dodecyl β-D-maltoside, heptanoyl-N-methylglucamide, n-heptyl-β-D-glucopyranoside, n-heptyl β-D-thioglucoside, n-hexyl β-D-glucopyranoside, nonanoyl-N-methylglucamide, n-noyl β-D-glucopyranoside, octanoyl-N-methylglucamide, n-octyl-β-D-glucopyranoside, octyl β-D-thioglucopyranoside, lysozyme, a PEG derivatized phospholipid, PEG derivatized cholesterol, a PEG derivatized cholesterol derivative, PEG derivatized vitamin A, PEG derivatized vitamin E, and random copolymers of vinyl acetate and vinyl pyrrolidone.

28. The method of claim 26, wherein the at least one cationic surface stabilizer is selected from the group consisting of a polymer, a biopolymer, a polysaccharide, a cellulosic, an alginate, a nonpolymeric compound, and a phospholipid.

29. The method of claim 26, wherein the at least one surface stabilizer is selected from the group consisting of cationic lipids, benzalkonium chloride, sulfonium compounds, phosphonium compounds, quarternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅dimethyl hydroxyethyl ammonium chloride, C₁₂₋₁₅dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl (ethenoxy)₄ ammonium chloride, lauryl dimethyl (ethenoxy)₄ ammonium bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, C₁₅ trimethyl ammonium bromides, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, POLYQUAT 10™, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, MIRAPOL™, ALKAQUAT™, alkyl pyridinium salts; amines, amine salts, amine oxides, imide azolinium salts, protonated quaternary acrylamides, methylated quaternary polymers, cationic guar, polymethylmethacrylate trimethylammonium bromide, polyvinylpyrrolidone-2-dimethylaminoethyl methacrylate dimethyl sulfate, hexadecyltrimethyl ammonium bromide, poly (2-methacryloxyethyltrimethylammonium bromide) (S1001), poly(N-vinylpyrrolidone/2-dimethylaminoethyl methacrylate) di methylsulphate quarternary (S1002), and poly(2-methylacryloxyamidopropyltrimethylammonium chloride) (S1004).

30. A method of treating a subject comprising administering to the subject an effective amount of a nanoparticulate insulin composition, wherein the composition comprises:
(a) insulin particles having an effective average particle size of less than about 5 microns prior to inclusion in the dosage form; and
(b) at least one surface stabilizer adsorbed on the surface of the insulin particles.

31. The method of claim 30, wherein the effective average particle size of the insulin particles is selected from the group consisting of less than about 4 microns, less than about 3 microns, less than about 2 microns, less than about 1500 nm, less than about 1 micron, less than about 800 nm, less than about 700 nm, less than about 600 nm, less than about 500 nm, less than about 400 nm, less than about 300 nm, less than about 200 nm, less than about 100 nm, less than about 75 nm, and less than about 50 nm.

32. The method of claim 30, wherein the concentration of insulin is selected from the group consisting of from about 99.5% to about 0.001%, from about 95% to about 0.1%, and from about 90% to about 0.5%, by weight, based on the total combined dry weight of the insulin particles and at least one surface stabilizer, not including other excipients.

33. The method of claim 30, wherein the concentration of the at least one surface stabilizer is selected from the group consisting of from about 0.001 to about 99.5%, from about 0.1 to about 95%, and from about 0.5 to about 90%, by weight, based on the total combined dry weight of the insulin particles and at least one surface stabilizer, not including other excipients.

34. The method of claim 30, wherein the insulin is in a form selected from the group consisting of crystalline particles, semi-crystalline particles, semi-amorphous particles, amorphous particles, and a mixture thereof.

35. The method of claim 30, wherein the composition further comprises at least two surface stabilizers.

36. The method of claim 16, wherein the at least one surface stabilizer is selected from the group consisting of a nonionic surface stabilizer, an anionic surface stabilizer, a cationic surface stabilizer, and an ionic surface stabilizer.

37. The method of claim 36, wherein the at least one surface stabilizer is selected from the group consisting of cetyl pyridinium chloride, gelatin, casein, phosphatides, dextran, glycerol, gum acacia, cholesterol, tragacanth, stearic acid, stearic acid esters and salts, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hydroxypropyl methylcellulose, carboxymethylcellulose sodium, methylcellulose, hydroxyethylcellulose, hydroxypropylmethyl-cellulose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers, poloxamines, a charged phospholipid, dimyristoyl phophatidyl glycerol, dioctylsulfosuccinate, dialkylesters of sodium sulfosuccinic acid, sodium lauryl sulfate, alkyl aryl polyether sulfonates, mixtures of sucrose stearate and sucrose distearate, triblock copolymers of the structure: -(-PEO)—(-PBO—)—(-PEO—)—, p-isononylphenoxypoly-(glycidol), decanoyl-N-methylglucamide; n-decyl β-D-glucopyranoside, n-decyl β-D-maltopyranoside, n-dodecyl β-D-glucopyranoside, n-dodecyl β-D-maltoside, heptanoyl-N-methylglucamide, n-heptyl β-D-glucopyranoside, n-heptyl β-D-thioglucoside, n-hexyl β-D-glucopyranoside, nonanoyl-N-methylglucamide, n-noyl β-D-glucopyranoside, octanoyl-N-methylglucamide, n-octyl-β-D-glucopyranoside, octyl β-D-thioglucopyranoside, lysozyme, a PEG derivatized phospholipid, PEG derivatized cholesterol, a PEG derivatized cholesterol derivative, PEG derivatized vitamin A, PEG derivatized vitamin E, and random copolymers of vinyl acetate and vinyl pyrrolidone.

38. The method of claim 36, wherein the at least one cationic surface stabilizer is selected from the group consisting of a polymer, a biopolymer, a polysaccharide, a cellulosic, an alginate, a nonpolymeric compound, and a phospholipid.

39. The method of claim 36, wherein the at least one surface stabilizer is selected from the group consisting of cationic lipids, benzalkonium chloride, sulfonium compounds, phosphonium compounds, quarternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅dimethyl hydroxyethyl ammonium chloride, C₁₂₋₁₅dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl (ethenoxy)₄ ammonium chloride, lauryl dimethyl (ethenoxy)₄ ammonium bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, C₁₅ trimethyl ammonium bromides, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, POLYQUAT 10™, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, MIRAPOL™, ALKAQUAT™, alkyl pyridinium salts; amines, amine salts, amine oxides, imide azolinium salts, protonated quaternary acrylamides, methylated quaternary polymers, cationic guar, polymethylmethacrylate trimethylammonium bromide, polyvinylpyrrolidone-2-dimethylaminoethyl methacrylate dimethyl sulfate, hexadecyltrimethyl ammonium bromide, poly (2-methacryloxyethyltrimethylammonium bromide) (S1001), poly(N-vinylpyrrolidone/2-dimethylaminoethyl methacrylate) di methylsulphate quarternary (S1002), and poly(2-methylacryloxyamidopropyltrimethylammonium chloride) (S1004).
